(19) United States
(12) Patent Application Publication
(10) Pub. No.: US 2014/0155761 A1
(43) Pub. Date: Jun. 5, 2014

(54) PHYSIOLOGICAL DETECTION DEVICE

(71) Applicant: CHIEN-YUAN YANG, New Taipei City (TW)

(72) Inventor: CHIEN-YUAN YANG, New Taipei City (TW)

(21) Appl. No.: 13/693,060

(22) Filed: Dec. 4, 2012

(51) Int. Cl. A61B 5/0205; A61B 5/00; A61B 5/11

(52) U.S. Cl. CPC A61B 5/0205; A61B 5/1117; A61B 5/746; A61B 5/6801; A61B 5/0002
USPC 600/483

(57) ABSTRACT

The physiological detection device is for equipping on a user, and contains a physiological signal module, an impact module, and a tracking module. The impact module, the physiological signal module, and the tracking module are data-linked to each other. The physiological signal module and the tracking module are also data-linked to at least an alarm module. When the physiological signal module detects some abnormality, an alarm is automatically generated from the alarm module. When the impact module detects some abnormality, an alarm will not be generated immediately. Instead the tracking module monitors the physiological signal module for a period of time. In this way, the false alarm due to that the impact module detects abnormality whereas the user is actually all right. The present invention as such achieves automatic detection and avoids false alarm.

### TECHNICAL FIELD OF THE INVENTION

The present invention generally relates to physiological detection device, and especially relates to physiological detection device which is capable of automatic detection and prevents false alarm.

### DESCRIPTION OF THE PRIOR ART

Falling down can induce serious injury and consequences to the senior or disabled people such as decreasing of mobility, damaging soft tissue, and head injury, etc. In more serious cases, there could be fracture or paralysis. Additionally, the person who falls down probably cannot get up by himself or herself, and probably no one can help nearby. The person could suffer both physiologically and psychologically.

For adult, falling down might not be a serious problem. However, it is a serious issue for adult who has cardiovascular disease which is more popular among them recently. The cardiovascular disease has high mortality rate as it can take a patient's life if treatment is not provided in time. People of the highly risky cardiovascular disease have to monitor their physiological condition constantly. Most people periodically go to hospitals for examination but it takes a lot of time and household nursing care. It is not convenient for seniors who cannot travel without help, or live far from hospital, or have chronic disease.

Therefore an alarm device for detection of falling down is important. The alarm device contains a heartbeat detector for detecting pulse, and an acceleration detector for detecting acceleration, and an alarm element data-linked to the heartbeat detector and the acceleration detector. When some abnormality is sensed by the heartbeat or acceleration detector, the alarm device is activated. However, the sensitivity of the acceleration detector is a key factor. If the sensitivity is not enough, the acceleration detector may fail to detect the abnormality. If the sensitivity is too high, the acceleration detector may generate false alarm. Even a user interface is provided for manual operation to avoid false alarm, these interfaces require that user have a certain capability and knowledge to setup it. It is not adequate for dementia or disabled people.

### SUMMARY OF THE INVENTION

Therefore a novel physiological detection device is provided herein so as to obviate the foregoing shortcomings of prior arts.

The main objectives of the present invention are as follows.

First, the false alarm due to human error is avoided by automatic detection.

Secondly, the false alarm is prevented by increasing sensitivity and distinguishing capability.

To achieve the objectives, the physiological detection device is for equipping on a user, and contains modules. First, a physiological signal module has a cardiogram element producing cardiogram signal of the user. Second, an impact module detects the user's acceleration change, direction and data-linked to the physiological signal module. Third a tracking module links to the physiological signal module and the impact module. The physiological signal module and the tracking module are data-linked to at least an alarm module. When the physiological signal module detects some abnormality, an alarm automatically generate from the alarm module. When the impact module detects abnormality, an alarm does not generate immediately. Instead of sending an alarm, the tracking module monitors the physiological signal module for a period of time. If the physiological signal module during this period of time detects abnormality, the tracking module activates the alarm module to generate an alarm. It prevents false alarm when jumping over a gap, lying down too hard onto a bed, etc. The present invention as such resolves the prior art's issues about low accuracy, not appropriate to some types of user, etc., by automatic detection.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a block diagram showing the various functional units of a physiological detection device according an embodiment of the present invention.

FIG. 2 is a flow diagram showing the operation of the physiological detection device of FIG. 1.

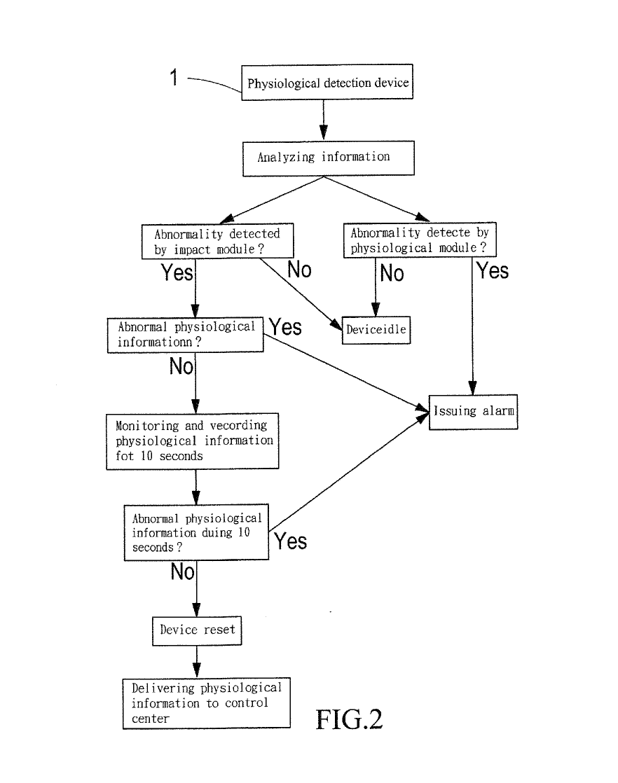

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

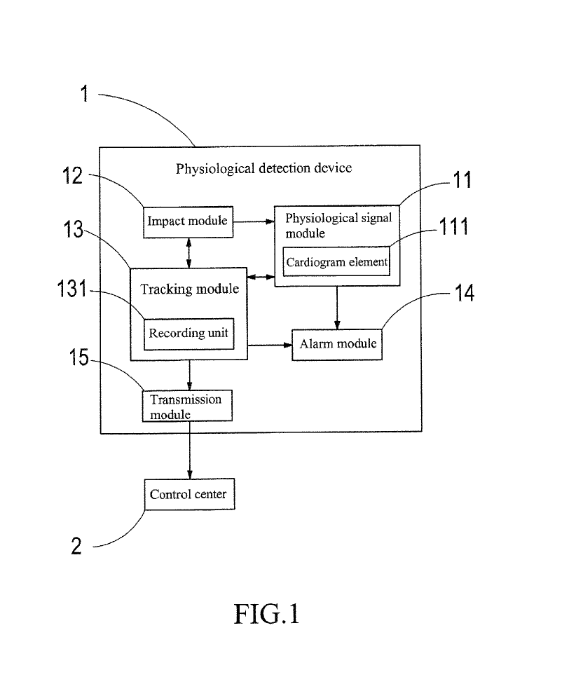

A physiological detection device 1 according to the present invention is worn by a user to collect physiological information and to detect abnormality for the user. As shown in FIG. 1, the physiological detection device 1 contains a physiological signal module 11, an impact module 12, and a tracking module 13. The physiological signal module 11 contains a cardiogram element 111 producing cardiogram signal of the user. The physiological signal module 11 and the impact module 12 are data-linked, and the impact module 12 is for detecting the user's acceleration change and direction. The impact module 12 can be a tri-axial accelerometer and there are various other embodiments for the impact module 12. The physiological signal module 11 and the impact module 12 are data-linked to the tracking module 13. The physiological signal module 11 and the tracking module 13 are data-linked to at least an alarm module 14. The tracking module 13 contains a recording unit 131 which is data-linked to a transmission module 15. The transmission module 15 in turn is data-linked to at least a control center 2.

The operation of the physiological detection device 1 is described as follows. As illustrated in FIGS. I and 2, after the physiological detection device 1 is started, information gathered from the physiological signal module 11 and the impact module 12 is analyzed. The information from the physiological detection device 1 is the physiological information such as the cardiogram signal of the user through the cardiogram element 111. If no abnormality is detected from the gathered information of the physiological signal module 11 or the impact module 12, the physiological detection device 1 remains idle. When the physiological signal module 11 detects some abnormality, an alarm is automatically generated from the alarm module 14. When the impact module 12 detects some abnormality, the impact module 12 will check first whether the physiological signal module 11 has detected abnormality. If the physiological signal module 11 has detected abnormality, an alarm is automatically generated from the alarm module 14. If the physiological signal module 11 hasn't detected abnormality, the impact module 12 automatically activates the tracking module 13. The tracking module 13 then monitors the physiological signal module 11 for a period of time (e.g., 10 seconds in the present embodiment) so as prevent false alarm. During this period of time, the recording unit 131 automatically stores the physiological information sensed by the physiological signal module 11. If the physiological information during this period of time indicates abnormality, the tracking module 13 activates the alarm module 14 to generate an alarm. If the physiological information during this period of time indicates no abnormality, the physiological detection device 1 resets itself. In the meantime, the recording unit 131 delivers the stored physiological information to the control center via the transmission module 15.

Compared to the prior art, the present invention has the following advantages.

First, because of the coordinated operations between the physiological signal module 11, the impact module 12, and the tracking module 13, false alarm can be prevented, thereby achieving enhanced detection capability

Second, also because of the coordinated operations between the physiological signal module 11, the impact module 12, and the tracking module 13, the sensitivity and differentiation capability of the impact module is increased, thereby achieving enhanced detection accuracy.

Third, through the coordinated operations between the physiological signal module 11, the impact module 12, and the tracking module 13, the physiological detection device is appropriate for various kinds of users, even including dementia or disabled people.

Fourth, by having the tracking module 13 to monitor the physiological signal module 11 automatically, manual interpretation to the physiological information is avoided, thereby achieving greater convenience.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A physiological detection device for equipping on a user, comprising a physiological signal module comprising a cardiogram element producing cardiogram signal of the user;
an impact module detecting the user's acceleration change and direction, the impact module data-linked to the physiological signal module; and
a tracking module data-linked to the physiological signal module and the impact module;
wherein the tracking module is activated by the impact module to monitor the physiological signal module for a period of time so as to avoid false alarm.

2. The physiological detection device according to claim 1, further comprising an alarm module data-linked to the physiological signal module and the tracking module.

3. The physiological detection device according to claim 1, wherein the tracking module comprises a recording unit.

4. The physiological detection device according to claim 3, wherein the recording unit is data-linked to at least a transmission module.

5. The physiological detection device according to claim 4, wherein the transmission module is data-linked to at least a control center.

6. The physiological detection device according to claim 1, wherein the impact module is a tri-axial accelerometer.
